(19) United States
(12) Patent Application Publication
(10) Pub. No.: US 2024/0408181 A1
(43) Pub. Date: Dec. 12, 2024

(54) PHARMACOLOGICAL COMPOUND FOR STIMULATION OF THE FOLLICULAR UNIT

(71) Applicant: NATURAL FOLIC, S.L., Granada (ES)

(72) Inventor: Antero FERNANDEZ SANCHEZ, Granada (ES)

(73) Assignee: NATURAL FOLIC, S.L., Granada (ES)

(21) Appl. No.: 18/665,934

(22) Filed: May 16, 2024

(30) Foreign Application Priority Data
Jun. 7, 2023 (ES) 202330473

(51) Int. Cl. A61K 38/39; A61K 31/07; A61K 31/122; A61K 31/198; A61K 31/4045; A61K 31/4188; A61K 31/506; A61K 31/58; A61K 31/59; A61K 33/30

(52) U.S. Cl. CPC A61K 38/39; A61K 31/07; A61K 31/122; A61K 31/198; A61K 31/4045; A61K 31/4188; A61K 31/506; A61K 31/58; A61K 31/59; A61K 33/30

(57) ABSTRACT

Pharmacological compound for stimulation of the follicular unit. The pharmacological compound is for the stimulation of the follicular unit. The pharmaceutical compound has fisnasteride, minoxidil, cystine, methionine, astaxanthin, melatonin, biotin, retinol, iron, selenium, collagen, zinc, molybdenum and vitamin D.

### BACKGROUND OF THE INVENTION

The present invention relates to pharmacological compounds, and more particularly, to a pharmaceutical compound for stimulation of the follicular unit whose purpose is to fight poor hair nutrition, both in healthy people with genetic disorders, and people with diseases where the hair is damaged by said disease.

The object of the present invention is to provide a product that stimulates the follicular unit in humans by pharmacological and natural resources in order to use them in seasonal or continuous effluvia, androgenic alopecia, secondary alopecia or poor vascularization, stress, and autoimmune processes.

The compound of the invention stimulates the follicular unit at a central level from the medulla with its own nutritional needs for correct formation of the external root epithelium.

The invention has been based on the study of hair needs for its correct nutrition, adapting the correlations between its components for a complete effectiveness.

### PRIOR ART

The maintenance of human hair has been one of the main interests of humanity since ancient times, several hair treatments have always been chosen in order to beautify and/or maintain the hear, so that the fight against alopecia has never been finished.

These types of treatments are applied from the exterior (where the follicular unit is not located) showings poor results in most cases.

Drugs have also been used for other specialty in which a minor hear improvement has been observed but without lasting clinical solution.

Until now, a truly effective and specific treatment against hair loss had not existed.

Applicant is not aware of any pharmacological compound for stimulation of the follicular unit having the novel features of the present invention.

### SUMMARY OF THE INVENTION

The present invention is a pharmacological compound for stimulation of the follicular unit, comprising:
Finasteride between 0.8 and 2.5 mg by mass;
Minoxidil between 0.25 and 5 mg by mass;
Cystine between 200 and 300 mg by mass;
Methionine between 50 and 150 mg by mass;
Astaxanthin between 4 and 12 mg by mass;
Melatonin between 0.2 and 1 mg by mass;
Biotin between 5 ng and 5 mg by mass;
Retinol between 300 and 800 ng by mass;
Iron between 6 and 14 mg by mass;
Selenium between 10ng and 150 mg by mass;
Collagen between 10 and 40 mg by mass;
Zinc between 5 and 10 mg by mass;
Molybdenum between 5 and 25 ng by mass;
Vitamin D between 50 and 100 ng by mass.

It is therefore one of the main objects of the present invention to provide a pharmacological compounds for stimulation of the follicular unit.

It is another object of this invention to provide a product

that stimulates the follicular unit in humans by pharmacological and natural compounds.

It is another object of this invention to provide a pharmacological compounds for stimulation of the follicular unit whose purpose is to fight poor hair nutrition, both in healthy people with genetic disorders, and people with diseases where the hair is damaged by said disease.

Further objects of the invention will be brought out in the following part of the specification, wherein detailed description is for the purpose of fully disclosing the invention without placing limitations thereon.

### DETAILED DESCRIPTION OF THE INVENTION

The present invention is a pharmacological compound for stimulation of the follicular unit. The proposed hair nutritional complex completely and satisfactorily resolves the previously mentioned problem.

For this purpose, more specifically, the product of the present invention comprises the following components:

### Finasteride

Finasteride is a drug approved by health authorities for androgenic alopecia. It prevents the conversion of testosterone to its most potent form, dihydrotestosterone (DHT), reducing plasma levels of this hormone. Its side effects are dose-dependent and reversible effect upon discontinuation of the drug. A meta-analysis performed to 17,634 people in 2016 indicates that it is not associated with significant alterations in sexual function.

### Minoxidil

Peripheral vasodilator that acts by relaxing the vascular smooth muscles, mainly at the arteriolar level. It is used to stimulate hair growth and reduce baldness. The biotransformation of absorbed minoxidil after topical application is not well known. It is believed that approximately 60% of the absorbed minoxidil is metabolized to minoxidil glucuronide, mainly in the liver. It was found that oral minoxidil was more effective than minoxidil lotion. During the study, no patient abandoned treatment and they had regular use of both options. The reasons why oral minoxidil is more effective than topical minoxidil are unknown to this day.

### Cystine

For a better understanding of its field of action, we have to study the hair fiber. Hair comprises a fibrous protein: alpha keratin. These keratins also have a special structure with intermediate filaments. These are aligned in such a way that they create the hair fiber.

A characteristic of alpha keratin is its high sulfur value. It is produced by the high content of di-amino acid cystine. Actually, the cysteine and methionine are the only natural amino acids containing sulfur. Its bonds may form disulfide bridges, in which the two cysteine molecules form the diamino acid cystine.

Knowing these cystine values, it is understood that, with an increase in the concentration of cystine in the hair follicles, an increase in the growth speed and thickness of the hair fibers is obtained.

### Methionine

It is an essential amino acid for hair growth. It is a powerful antioxidant and comprises sulfur, the key ingredient to counteract hair disorders. Due to its lipotropic function, it increases blood flow in the hair follicles, promoting greater hair growth.

### Astaxanthin

Its antioxidant power is 800 times more powerful than coenzyme Q10 and 6000 times more powerful than vitamin C. It is an antioxidant explosion for all cells.

The astaxanthin renews the skin and increases its health, improves the health of connective tissue, improves the health of healthy cell membranes after exposure to UV light, and helps to maintain an antioxidant balance.

### Melatonin

Melatonin is an hormone universally known for its power to regulate circadian rhythms and is also involved in hair growth. Until now, commonly, it was related to the property to facilitate sleep, but science has been investigating its relationship with hair for some time. According to the study of Melatonin and hair physiology performed by Dr. Massimo Milani, it have been demonstrated the antioxidant and anti-inflammatory activity of Melatonin in the skin and its relation in hair growth to stimulate its anagen phase and attenuate oxidative stress in the hair follicle.

### Biotin

Biotin improves the structure of the keratin, which is a basic protein in the hair, skin, and nails, therefore it can improve hair health, including volume and scalp coverage.

### Retinol

Vitamin A and its derivatives (retinoids) are important for the development and maintenance of multiple epithelial tissues, including skin, hair, and sebaceous glands. Serious effects are seen when there are deficiency or excess of vitamin A.

Increases blood flow to hair follicles and stimulates the formation of new blood vessels.

### Iron

Iron is the mineral responsible for producing hemoglobin, which are the proteins that transport oxygen through the body. One of its benefits is that it allows oxygen to be transported to the roots of the hair, which influences healthier and stronger hair.

### Selenium

Selenium is an anti-infective agent, which relieves itching and flaking of the scalp and removes the dry, flaky particles commonly called dandruff or seborrhea.

### Collagen

Collagen represents a fundamental ingredient for healthy and strong hair. This substance, which the body generates naturally, tends to disappear with age, making hair look dull, weak, and brittle.

The collagen keeps an strong and resistant hair. Because of its extra contribution of amino acids, hair receives the nutrients it needs to be healthy.

The collagen counteracts hair loss. The collagen provides nutrients to the root cells, stopping hair loss.

Helps accelerate growth. The hair grows faster because the improving of the circulation in the area.

Conclusion, the main advantage of taking collagen is that it acts by deeply regenerating hair.

### Zinc

To understand the relation between hair health and zinc, firstly it is needed to understand the connection between zinc insufficiency and hair loss. Lack of zinc is, in fact, the main cause of the deterioration of the structure of the protein that makes up the hair follicle. This weakening of the follicle can in turn cause hair deterioration and hair alopecia.

The zinc has also been shown to play a crucial role in the production of DNA and RNA, being necessary for hair follicle cell division and healthier hair growth.

At the same time, zinc helps keep balanced hormone levels, making it one of the essential reasons why it is considered so effective for prevent hair loss and the appearance of gray hair. Aging and graying hair can return to its original color when nourished with diets rich in zinc or supplements formulated with this element.

### Molybdenum

Molybdenum is responsible for the metabolism of sulfur amino acids, essential for the synthesis of keratin, particularly methionine.

Molybdenum is a mineral that people need to stay healthy. The body uses molybdenum to process proteins and genetic material such as DNA. Molybdenum also helps to destroy medications and toxic substances that enter into the body.

### Vitamin D

If the body does not have enough vitamin D, accelerated hair loss may occur, which would be related to alopecia.

This happens because the deficiency of this nutrient negatively influences the development of the hair growth cycle and is related to the appearance of autoimmune problems, since vitamin D contributes to the immune processes of the scalp being able to distinguish between bacteria and normal tissue.

By not having Vitamin D, an autoimmune reaction against this tissue occurs, which translates into weakness and vulnerability, altering the hair growth cycle, so the hair falls out prematurely and is much more difficult to return to grow.

There are also scientific studies that have proven that vitamin D has the particularity of regulating the expression of genes that promote normal growth of the hair follicle. In addition, it modulates the production of T lymphocytes, which are immune cells acting as guardians in defense of the human organism.

More specifically, the composition comprises the following elements:

| | |
|---|---|
| Finasteride | between 0.8 and 2.5 mg. |
| Minoxidil | between 0.25 and 5 mg. |
| Cystine | between 200 and 300 mg. |
| Methionine | between 50 and 150 mg. |
| Astaxanthin | between 4 and 12 mg. |
| Melatonin | between 0.2 and 1 mg. |
| Biotin | between 5 ng and 5 mg. |
| Retinol | between 300 and 800 ng. |
| Iron | between 6 and 14 mg. |
| Selenium | between 10 ng and 150 mg. |
| Collagen | between 10 and 40 mg. |
| Zinc | between 5 and 10 mg. |
| Molybdenum | between 5 and 25 ng. |
| Vitamin D | between 50 and 100 ng. |

The product of present invention is administered orally, with daily doses, either in liquid or solid form, in any form for oral ingestion, and can be obtained in tablets, dragees, lozenges, and hard or soft capsules.

In a preferred embodiment, the capsules may contain plant or animal material, as well as, glycerin for their coating.

According to the present invention, a highly effective hair nutritional complex is achieved for hair treatment.

### EXAMPLE OF THE INVENTION

As an illustrative but non-limitative example of the present invention, to obtain 276.25037 mg of hair nutritional complex, the formulation comprises the following:

| | | |
|---|---|---|
| Finasteride | 0.8 | mg. |
| Minoxidil | 0.25 | mg. |
| Cystine | 200 | mg. |
| Methionine | 50 | mg. |
| Astaxanthin | 4 | mg. |
| Melatonin | 0.2 | mg. |
| Biotin | 5 | ng. |
| Retinol | 300 | ng. |
| Iron | 6 | mg. |
| Selenium | 10 | ng. |
| Collagen | 10 | mg. |
| Zinc | 5 | mg. |
| Molybdenum | 5 | ng. |
| Vitamin D | 50 | ng. |

The composition of the present invention can be manufactured in liquid or solid form. Specifically, present invention may be in form of tablets, dragees, lozenges, and hard or soft capsules. Present invention provides a composition for stimulation of the follicular unit in humans using natural products to be able to use it in seasonal or continuous effluvia, androgenic alopecia, secondary alopecia or poor vascularization, stress, and autoimmune processes.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A pharmacological compound for stimulation of the follicular unit, comprising:
Finasteride between 0.8 and 2.5 mg by mass;
Minoxidil between 0.25 and 5 mg by mass;
Cystine between 200 and 300 mg by mass;
Methionine between 50 and 150 mg by mass;
Astaxanthin between 4 and 12 mg by mass;
Melatonin between 0.2 and 1 mg by mass;
Biotin between 5 ng and 5 mg by mass;
Retinol between 300 and 800 ng by mass;
Iron between 6 and 14 mg by mass;
Selenium between 10ng and 150 mg by mass;
Collagen between 10 and 40 mg by mass;
Zinc between 5 and 10 mg by mass;
Molybdenum between 5 and 25 ng by mass;
Vitamin D between 50 and 100 ng by mass.
